(19) United States
(12) Patent Application Publication
(10) Pub. No.: US 2009/0287172 A1
(43) Pub. Date: Nov. 19, 2009

(54) ABSORBENT ARTICLE WITH LEAKAGE WARNING SYSTEMS AND METHOD OF USE THEREOF

(76) Inventor: William Lowe, Los Angeles, CA (US)

Correspondence Address: TROJAN LAW OFFICES, 9250 WILSHIRE BLVD, SUITE 325, BEVERLY HILLS, CA, 90212, US

(21) Appl. No.: 12/121,980

(22) Filed: May 16, 2008

(51) Int. Cl. A61F 13/42

(52) U.S. Cl. 604/361

(57) ABSTRACT

The present invention includes an absorbent article, more specifically, an absorbent article including a leakage warning system. The leakage warning system is located along the outer perimeter of the absorbent article. Fluids activate the leakage warning system. Thus, when fluids reach the outer perimeter of the absorbent article, the leakage warning system is activated. Once activated, the leakage warning system provides a warning signal that fluids have reached the edge of the absorbent article. In one embodiment, the leakage warning system is a liquid-activated band. Once activated, the band provides a temperature-change sensation to the user to signal that the fluids have reached the edge of the absorbent article and thus warns a user of possible leakage before it actually occurs.

### FIELD OF THE INVENTION

The present invention relates to an absorbent article. More specifically, the invention relates to an absorbent article, such as a sanitary napkin, with a leakage-warning system and a method of use thereof.

### BACKGROUND OF THE INVENTION

Absorbent articles designed to absorb fluids discharged from the human body, such as sanitary napkins and panty liners, are well known. Many different constructions of absorbent articles have been made to protect a user's clothing from being soiled by discharge of such fluids. However, sanitary napkins described in the prior art do not provide a method to warn users when the absorbent article has reached, or is approaching capacity. As a result, discharged fluids can leak beyond the edge of the sanitary napkin or panty liner without the user knowing.

There have been several attempts to prevent leakage in absorbent articles. Sanitary napkins with wings extending from the side edges of the pad are well known in the art. Another method to prevent side leakage is described in U.S. Pat. No. 5,817,079, in which discreet areas of fluid-repellent materials are placed in the sanitary napkin to provide barriers to bodily fluid leakage from the product. Similarly, Canadian Patent No. 884,608 describes treating the edges of a sanitary napkin with hydrophobic material to prevent side leakage. U.S. Pat. No. 5,624,423 describes a sanitary napkin that includes a barrier means encircling an absorbent protuberance to prevent leakage. These prior art references help to prevent leakage but do not teach sanitary napkins that warn users before leakage occurs.

Other prior art references describe absorbent articles that detect fluids. U.S. Pat. No. 5,647,863 describes a sanitary napkin that provides a signal by way of an indicator member that becomes noticeably stained when the storage capacity of the sanitary napkin is substantially exhausted. This type of fluid detection does not provide a specific and clear signal to the user that fluid has reached the edge of the sanitary napkin, warning the user that leakage may occur.

U.S. Pat. No. 5,728,125 describes a sanitary napkin having a temperature-sensitive reactive chemical product that responds by turning cold when it contacts and dissolves in menstrual flow. This sanitary napkin's fluid detection, however, is activated when any fluid reaches the pad, and only indicates to the user that her menstrual flow has begun. The fluid chemical product is not strategically placed on the perimeter of the pad and therefore cannot warn the user before fluids reach the edge of the pad to prevent leakage over the edge of the pad.

Thus, what is needed is a sanitary napkin involving a warning system to detect fluids at the periphery of the pad and provide a signal, such as a temperature change, to warn the user before a leakage occurs. Such an absorbent article will provide a greater peace of mind to women, as women would no longer have to think about whether or not the pad requires changing before it embarrassingly leaks onto their outer clothing. With these goals in mind, the inventor sought to create an easy to use absorbent article that incorporates a clear warning signal that is detected by the user, to warn the user when fluids reach the edge of the absorbent article and thereby prompt the user to take action (inspect, change the article, etc.) and prevent leakage.

### SUMMARY OF THE INVENTION

The present invention describes an absorbent article with a leakage-warning system. The absorbent article includes a leakage warning system formed along the outer perimeter of the absorbent article, whereby the leakage warning system activates when bodily fluids reach it and provides a signal to a user, thereby warning the user when bodily fluids reach the perimeter of the absorbent article, and the user may act upon the warning to prevent leakage.

In one embodiment, the absorbent article comprises a body-facing liquid permeable top sheet having an outer perimeter, a garment-facing liquid impermeable back sheet, an absorbent core disposed between said top sheet and said back sheet, said absorbent core having an outer perimeter, and a leakage warning system, wherein the leakage warning system is formed along either the outer perimeter of the top sheet or the outer perimeter of the absorbent core. The back sheet can be coated adhesive so that it can be attached with a garment worn by the user. The leakage warning system is activated when bodily fluids reach the outer perimeter and thereby provides a signal to the user that warns the user when bodily fluids reach the periphery of the absorbent article.

In one aspect, the absorbent article is a feminine hygiene product selected from a group consisting of panty liners, sanitary napkins, incontinence pads, and breast pads.

In another aspect, the leakage warning system is comprised of a liquid-activated band, whereby the liquid-activated band activates when bodily fluids reach it and it provides a signal to a user, thereby warning said user when said bodily fluid reaches the perimeter of said absorbent article to prevent leakage.

In a further aspect, the signal is comprised of a temperature-change signal selected from a group consisting of a warm, cold, and hot temperature sensation.

In yet another aspect, the liquid-activated band is comprised of an endothermic material or exothermic material.

In yet another aspect, the signal is comprised of a sensory feedback signal selected from a group of tingling, compression, contraction and vibrating sensations.

In a further aspect, the leakage warning system has an outer perimeter comprised of a material selected from a group consisting of a semi-permeable and impermeable material, whereby the material is capable of slowing or stopping the flow of liquids past said outer perimeter of said leakage warning system.

A method for using the absorbent article is also described.

### BRIEF DESCRIPTION OF THE DRAWINGS

FIG. 1 is a top perspective view of an absorbent article having a leakage warning system according to the present invention;

FIG. 2 is a cross-sectional side perspective view of an absorbent article having a leakage warning system according to the present invention;

FIG. 3 is a top orthogonal view of an absorbent article having a leakage warning system according to the present invention;

FIG. 4 is a top orthogonal view of an absorbent article having a leakage warning system according to the present invention; and

FIG. 5 is a top orthogonal view of an absorbent article having a leakage warning system according to the present invention.

FIG. 6 is a top orthogonal view of an absorbent article having a leakage warning system according to the present invention.

FIG. 7 is a top orthogonal view of an absorbent article having a leakage warning system according to the present invention.

FIG. 8 is a top orthogonal view of an absorbent article having a leakage warning system according to the present invention.

FIG. 9 is a cross-sectional side perspective view of an absorbent article having a leakage warning system according to the present invention.

### DETAILED DESCRIPTION OF THE INVENTION

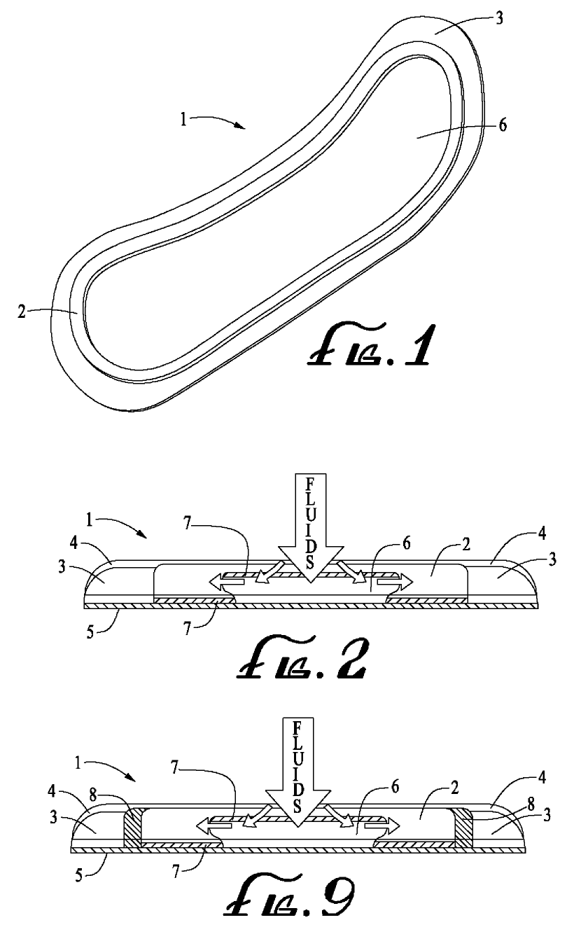

The present invention includes an absorbent article, more specifically, a feminine hygiene product, such as a sanitary napkin or panty liner, having a leakage warning system that provides a warning signal to the user, and is only perceived by the user, when fluids reach the edge of the absorbent article or when the absorbent article reaches capacity. A non-limiting example of the absorbent article (1) is shown in FIG. 1, in the form of a sanitary napkin. The absorbent article (1) can be in the form of other feminine hygiene products, including panty liners and incontinence pads. The article can be in the form of an attachment or insert and can have various shapes to contour the body and undergarments, for example, thong, extra-long, or styles with or without extra-protective side wings. The different shapes are provided to accommodate the individual user's needs. The absorbent article can also be in the form of a breast pad to absorb excess bodily fluids discharged during breastfeeding. The absorbent article (1) includes a leakage warning system (2) that can be located on or embedded in different parts of the absorbent article (1). For example, the leakage warning system (2) can be located on the outer perimeter (3) of a body-facing liquid permeable top sheet (4) of the absorbent article (1). The leakage warning system (2) can also be located on the outer perimeter (3) within the absorbent core (6) of the absorbent article (1) (not shown).

As illustrated in FIG. 2, the absorbent article (1) comprises a garment-facing liquid impermeable back sheet (5) and an absorbent core (6) disposed between said top sheet (4) and said back sheet (5). When a bodily fluid, such as blood, reaches the leakage warning system (2), the leakage warning system (2) is activated and provides a signal to the user. The leakage warning system (2), in one embodiment, is comprised of a liquid-activated band. In this embodiment, the liquid-activated band, located at the outer perimeter (3) of the absorbent article (1), reacts with the fluid that comes into contact with the band and provides a signal to the user. This specific location of the leakage warning system (2) (for example, the liquid-activated band), allows the absorbent article (1) to provide a distinct signal to the user specifically when bodily fluids reach the periphery of the absorbent article (1) and hence provides a distinct signal to the user before leakage can occur.

As a non-limiting example, the signal can be a tingling sensation. The tingling sensation is activated when fluids reach the liquid activated band and thus warns the user to change the absorbent article before leakage occurs. In such an embodiment, non-toxic materials known in the art to produce such tingling sensations comprise the liquid activated band. Alternatively, the leakage warning system can be comprised of wireless or Bluetooth™ technology to provide an electronic signal to a remote device when activated by liquids.

As another non-limiting example, the signal is a temperature change. When fluid reaches the leakage warning system (2) (for example, the liquid-activated band), the fluid activates the leakage warning system (2) and the leakage warning system (2) changes the temperature of the bodily fluids, thereby signaling to the user that the fluids have reached the outer perimeter (3) of the absorbent article (1) before leakage can occur. The leakage warning system (2) may be comprised of material (7) that is exothermic or endothermic. For example, an exothermic material, upon activation by bodily fluids, provides a warm sensation to the user, which is only perceived by the user. In the alternative, an endothermic material, upon activation by bodily fluids, provides a cold sensation to the user that is only perceived by the user.

As known in the art, the endothermic or exothermic materials can be particles that absorb or release heat when dissolved in aqueous solutions, including: xylitol; salt hydrates, such as sodium acetate, sodium carbonate, sodium thiosulfate, and sodium hyposulfite; anhydrous salts, such as potassium nitrate, ammonium chloride, potassium chloride, and sodium nitrate; and aluminum chloride, or the like. Also known in the art, the endothermic or exothermic materials can be substances that absorb or release heat upon reaction with an aqueous solution, including ketals, such as menthone ketals. The endothermic and exothermic materials can be bio-degradable, environmentally friendly and are non-hazardous materials known in the art.

The leakage warning system (2) can be further comprised of a semi-permeable or impermeable material (8), as shown in FIG. 9. The semi-permeable or impermeable material (8) is formed on the outer perimeter of the system (2), for example, surrounding the liquid-activated band. Such placement of the semi-permeable or impermeable material (8) will impede the flow of liquids once liquids reach the leakage warning system, and slow or stop the liquid from flowing past the leakage warning system (2). The material (8) provides an extra barrier beyond the warning system (2) to help prevent leakage.

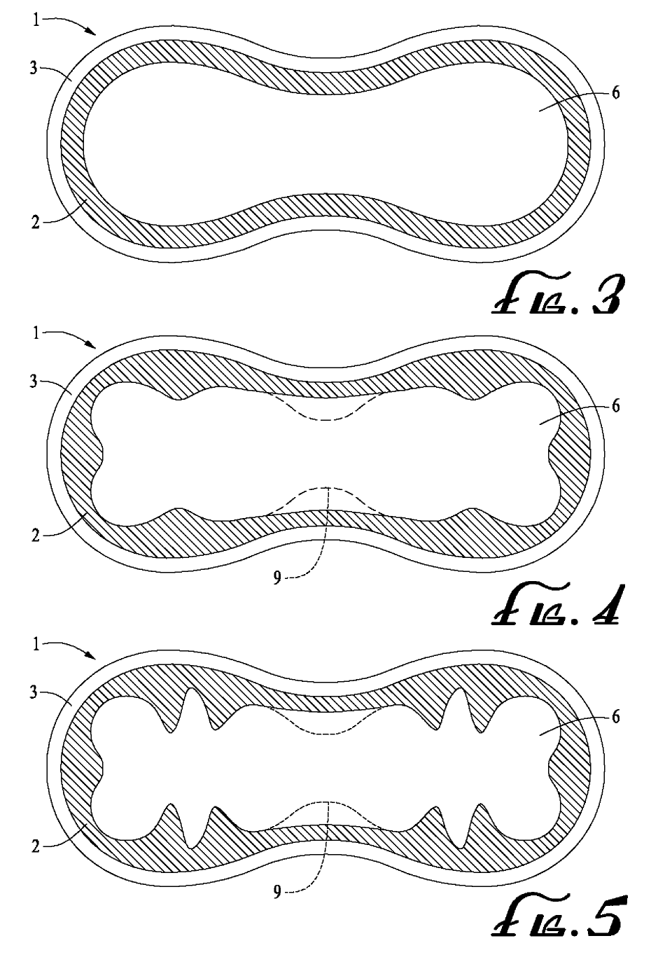

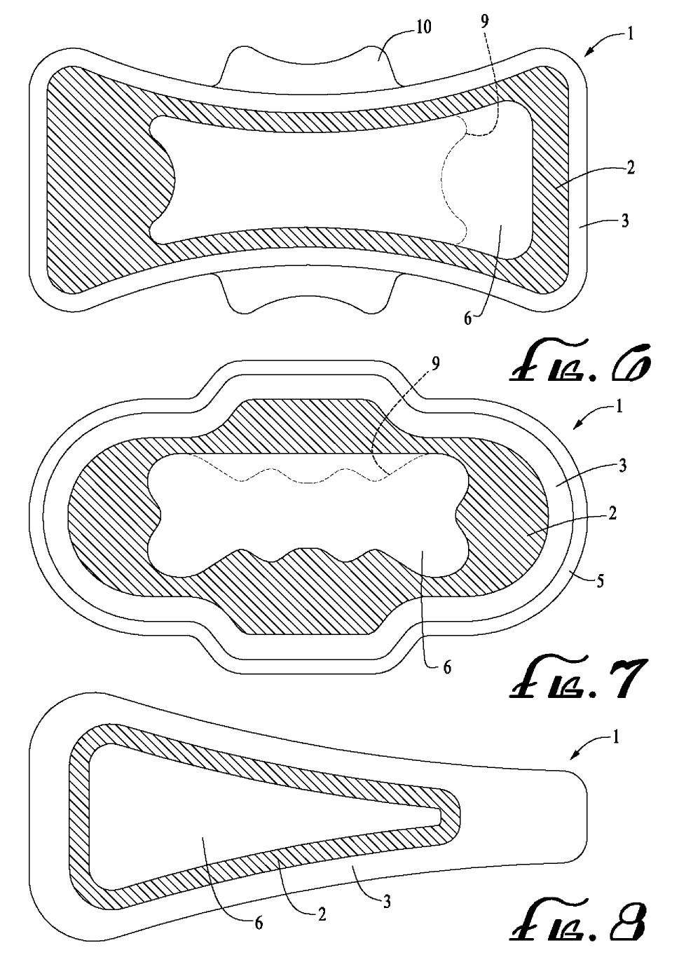

FIGS. 3, 4, 5, 6, 7 and 8 illustrate different embodiments of the leakage warning system (2) along the outer perimeter (3) of the body-facing liquid permeable top sheet (4) of the absorbent article (1). In these illustrations, leakage warning systems (2), particularly in the form of differently shaped liquid-activated bands, are shown. As a non-limiting example, the absorbent article (1) can include quilted patterns (9) for added absorption and comfort. FIGS. 6 and 7 illustrate that the absorbent article (1) can include wings (10) for extra leakage protection on the sides of the absorbent article (1). FIG. 8 shows an example of the absorbent article (1) in a thong shape.

While the foregoing describes the present invention in relation to illustrations and examples, it is understood that it is not intended to limit the scope of the invention to the illustrations and examples described herein. On the contrary, it is intended to cover all alternative modifications and equivalents that may be included in the spirit and the scope of the invention as defined by the appended claims.

What is claimed is:

1. An absorbent article having a leakage warning system, comprising:
a body-facing liquid permeable top sheet having an outer perimeter;
a garment-facing liquid impermeable back sheet;
an absorbent core disposed between said top sheet and said back sheet, said absorbent core having an outer perimeter; and
a leakage warning system, wherein said leakage warning system is formed along an outer perimeter of said absorbent article,
whereby said leakage warning system is activated when bodily fluids reach said leakage warning system formed along said outer perimeter and provides a signal to a user, thereby warning said user when said bodily fluid reaches said outer perimeter of said absorbent article to prevent leakage.

2. The absorbent article of claim 1, said absorbent article is a feminine hygiene product selected from a group consisting of a panty liner, a sanitary napkin, an incontinence pad, and breast pad.

3. The absorbent article of claim 1, wherein said leakage warning system is comprised of a liquid-activated band, whereby said liquid-activated band is activated when bodily fluids reach said liquid-activated band formed along said outer perimeter and provides a signal to a user, thereby warning said user when said bodily fluid reaches said outer perimeter of said absorbent article to prevent leakage.

4. The absorbent article of claim 1, wherein said leakage warning system is further comprised of an outer perimeter, wherein said outer perimeter is comprised of a material selected from a group consisting of a semi-permeable and impermeable material, whereby said material slows or stops the flow of liquids past said outer perimeter of said leakage warning system.

5. The absorbent article of claim 1, wherein said signal is comprised of a temperature-change signal selected from a group consisting of a warm, cold, and hot temperature sensation.

6. The absorbent article of claim 3, wherein said liquid-activated band is comprised of a material selected from a group consisting of an endothermic and exothermic material.

7. The absorbent article of claim 1, wherein said signal is comprised of a sensory feedback signal selected from a group of tingling, compression, contraction and vibrating sensations.

8. The absorbent article of claim 1, wherein said signal is comprised of an electronic signal.

9. The absorbent article of claim 3, wherein said liquid-activated band is comprised of a material capable of producing a sensory feedback signal.

10. The absorbent article of claim 1, wherein said wherein said back sheet further comprises a surface coated with adhesive whereby said back sheet can be attached with a garment.

11. A method for using an absorbent article having a leakage warning system formed along an outer perimeter of said absorbent article, comprising the acts of:
attaching said absorbent article to a garment so that said absorbent article is capable of absorbing bodily fluids;
detecting a signal when said bodily fluids contact said leakage warning system formed along said outer perimeter of said absorbent article; and
removing said absorbent article when said signal is detected, whereby a user avoids leakage of said bodily fluids from said absorbent article.
